(19) United States
(12) Patent Application Publication
(10) Pub. No.: US 2018/0151252 A1
(43) Pub. Date: May 31, 2018

(54) METHOD AND APPARATUS FOR A VIRTUAL CLINICAL TRIAL SELF-RECRUITMENT MARKETPLACE FOR PATIENTS BASED ON BEHAVIORAL STRATIFICATION, PATIENT ENGAGEMENT AND PATIENT MANAGEMENT DURING CLINICAL TRIALS USING BEHAVIORAL ANALYTICS, GAMIFICATION AND COGNITIVE TECHNIQUES

(71) Applicant: OPTIMAL STRATEGIX GROUP, Newtown, PA (US)

(72) Inventors: Giri IYER, Marietta, GA (US); Hua FAN, Cumming, GA (US); Ramamirtham SUKUMAR, Princeton, NJ (US)

(21) Appl. No.: 15/580,267

(22) PCT Filed: Jun. 7, 2016
(86) PCT No.: PCT/US2016/036235

§ 371 Date: Dec. 6, 2017

(51) Int. Cl. G16H 10/20; G06Q 40/00

(52) U.S. Cl. CPC G16H 10/20; G16H 10/60; G06Q 40/00

(57) ABSTRACT

Method, Apparatus and non-transitory computer readable media that enhance patient-recruitment and participation in clinical trials preferably includes structure and/or steps whereby one or more APIs are used to interface at least one Cognitive Analytics Value Inference and Intelligence-Healthcare (CAVII-H) server with the patient(s), Pharma participant(s), Contract Research Organization(s), Trial Investigator(s), and Healthcare Providers (such as a physician). The at least one CAVII-H server preferably use at least one of stored trial data, user medical data, user behavioral date, user application collected data user registration data, healthcare professional data to guide patient participation. Preferably, information is obtained from the users through plural interactive algorithms designed to sharpen and perfect patient selection, preferably throughout the life of each trial. This creates a global clinical-trial patient engagement, recruitment and retention marketplace, which is open to patents and all trial stakeholders.

### BACKGROUND OF THE INVENTION

### 1. Field of the Invention

The present invention relates to a predictive technique and tool for creating a global virtual marketplace for patient self-recruitment and management for the purposes of Pharma and medical device research. The marketplace system delivers superior patient engagement based on proactive patient self-stratification by clinical, behavioral, social and financial indices that secures higher recruitment rate and lower patient attrition during human clinical trials using an ASEMAP™ (Adaptive Self Explicative Multiple attribute preference models) tool and other gamification and cognitive analysis techniques.

### 2. Description of Related Art

On average, Pharma companies spend >$2B over 10-12 years on the R&D of a drug and often have high failure rates, particularly during early stages of the clinical process. This includes an estimated cost of between $50,000-$500,000 per patient per clinical trial. Despite these costs, the Pharma companies and their trial intermediaries secure only a retention rate of 48% with their recruited patients, and every time a patient drops out of the clinical trial, the sponsoring company loses significant amounts of money balancing the trial cohort base again to eliminate bias across trial arms and to restore the power of the trial. Today 80 percent of all trials are behind their patient recruitment goals and the industry greatly needs new innovation.

Historically, human clinical trials have been researcher led and designed for research integrity and not patient comfort. These researchers developed clinical protocol by disease for patient screening and selection including rigorous clinical inclusion and exclusion criteria, which are typically based extensively on clinical indicators and outcome information. Clinical sites then diligently use this clinical protocol to select cohorts of patients to match targeted study profiles to patients, one patient at a time through a systematic screening process for each study. The exclusion criteria are used to screen out ineligible patients without understanding their behavioral attributes.

The patient is not authorized to select himself or herself into a screening pool and opt-in proactively to be additionally screened based on the inclusion criteria. The inclusion process of today is clinical researcher-initiated and patients generally do not get to assess for themselves easily whether the study is right for them without much research and drive and the protocol generally does not accommodate for patients who display a higher sense of behavioral urgency to participate in the research. Once patients are in the trial, some will sadly drop out due to adverse events and some due to changing trial protocol redesigned to adapt to research and clinical realities. Little to no attention is paid to behavioral reasons that drive 50 percent of the patients to drop out of studies before the trial completes. Some of the reasons for patient dropouts are
Excessive testing of patient
Documentation burden on the patient or their caregiver
Repetitive and redundant visits that are difficult to commit to for a sickly patient
Long travel trips to clinical sites and associated discomfort
Lack of ability to share information from home using their own devices
Duration of the trial itself,
Painful treatment delivery mechanisms like intravenous or injections or frequency of doses
Fatigue or other side effects that they do not tolerate well
Adverse Reactions and complexities that render them ineligible or unable to continue participation in the trial
Lack of improvement in their disease journey and feeling that the participation is not getting them any benefits in terms of therapeutics or even information.
Painful and highly repetitious, bureaucratic data gathering processes including long questionnaires
Unforeseen delays in prompt compensation for patient time and participation in the study,
Lack of motivation or disengagement due to emotional or social issues

These factors are Clinical, Social, Behavioral and Financial that contribute to a patient's engagement or disengagement with the trial. Studies have shown that providing timely and accurate information about the trial and its progress may improve the patient involvement and lower the attrition rates.

U.S. Pat. No. 7,711,580 B1, to Hudson discloses a system and method for matching patients with clinical trials and particular trial sites, prequalifying patients for clinical trials and trial sites, and providing information to patients to allow them to inform themselves about available clinical trials and trial sites. The method comprises receiving patient profile information for a patient at a server connected to a computer network, the patient profile information submitted by a user at a terminal connected to the network, comparing the patient profile information with acceptance criteria for clinical trials stored in a database, the comparison performed by the server, determining whether the patient prequalifies for any clinical trials.

U.S. Pat. No. 7,904,313 B2 to Knight discloses techniques for recruiting a patient into a clinical trial, including receiving patient-specific data from a remote network device at a server, accessing criteria of more than one clinical trial at the server and determining one or clinical trials having criteria satisfied by the patient specific data. The system comprises of collecting patient specific data from patient interface, in comparison to the set of disease specific data to generate a set of patient-disease characteristics; compare the set of patient-disease characteristic to a set of trial-specific criteria corresponding to the clinical trial and determine whether the match exists between patient and clinical trial.

Current related art focuses only on system or methodology of clinical researchers and sites recruiting patients by matching patient-disease characteristics only with the trial specific criteria but do not take other factors into account such as Social, Financial and Behavioral that not only provide better matching criteria but also drive patient's engagement with the trial. Also, the current art is driven by Clinical Research Sponsors with patients being just passive participants. Once the patient data is collected through the questionnaire, he/she has a limited role in the process of matching and selection of the trial, which results in a low or complete lack of engagement with the trial. In today's era, these same patients can book their own flights, hotels, cars, etc. but not interact with trials that may benefit them where they are contributing their body to the research itself and are researchers themselves. This paradigm of not trusting the patient creates an inefficient system that makes the cost of the therapy itself expensive to the healthcare systems of the world.

### SUMMARY OF THE INVENTION

Thus, in view of the above, what is needed is an interconnected analytical platform and/or system that enables the creation of a global patient engagement, recruitment and retention marketplace. This marketplace can help patients create their own profiles and potentially self-recruit themselves into trials. This is a notable advantage of the disclosed embodiments. The marketplace enables patients to self-segment and stratify themselves based on their predicted engagement urgency index that includes behavioral indices; that provide the patients a list of better matching trials, enable them to self-recruit, and then keep them engaged through the trial life cycle by providing them easy to use self-service tools and accurate and timely information dissemination to the care team as well as the research team. In accordance with an embodiment of the invention, the patient is provided with these self-reporting quality of life and self-assessment forms, surveys, documents and updates on a mobile device, such as a smart phone or a tablet. Being able to fill up these surveys and automate data remotely goes a long way to get the patient to be better compliant with the trial protocol, and even participate in the trial completely from home on some occasions. This could usher in a world of virtual clinical trials where trials are then completed faster, better, cheaper from home whenever possible and the resultant economic benefits are passed on to the patients themselves as lower drug/treatment costs worldwide.

It is an advantage of the present invention to overcome the problems of the related art and to provide a system that puts patient at the center of the clinical studies as a researcher rather than forcing the patient to adapt to a rigorous and bureaucratic research process, which is also done one trial at a time by each Pharma or research organization. The system comprises a sophisticated back-end data analytics platform that is preferably cloud-based and supports multiple applications that are provisioned for secure and interactive views by multiple stakeholders like patients, Pharma and Medical Device companies, contract research organizations and research sites, and other intermediaries and regulatory authorities. Notable in the present embodiments is a preferably free patient-centric mobile application CAVII-H™ (Cognitive Analytics Value Inference and Intelligence-Healthcare) that is available to patients worldwide and is preferably available on Apple iOS, Google Android and the web. CAVII-H™ preferably, facilitates the patients to create his or her own 360-degree profile. The application preferably creates a singular auto-updating continuous data stream including day-to-day health data from personal health monitoring devices, application programming interfaces (API) to collect patient clinical, financial and social media related data, questionnaires and other tools to collect patient behavior or personality related data. APIs/web services are preferably used to collect clinical trials data, analytical algorithms to profile and segment patients to get matching clinical trials (for example: clinicaltrials.gov and its equivalent web sites worldwide), and Clinical Trials Sponsor application(s) to monitor and manage patients and clinical trials. The patient is given multiple and granular ability to control their own data and consent to share this data (with their explicit and repeated informed consent) to share with researchers and other players in this marketplace, seamlessly. This eliminates a lot of friction and inefficiency in the marketplace and creates a growing pool of patients including those who are in the shadows and dormant in the large and rapidly growing social media community like Facebook, Twitter, etc.

An overall predictive urgency index is preferably derived from patient's clinical, social, behavioral and financial indexes, and would help Clinical Trial Sponsors to take corrective actions for patients with a low engagement urgency index and to help them make better informed decisions before they drop out of the trial. The predictive urgency index also lets a patient know that he/she may not be a behavioral fit for these trials and his/her likelihood of success is lower strictly on behavioral index scores. Elimination of patients on this ground would optimize selection to those who are likely to perform well on the trial protocol. This is clearly a first in the industry and may create significant disruption since it creates new statistical challenges for researchers who are now forced to reckon with patient preferences in their trial designs up front rather than at the tail end of the process where patient participation is weak. 80% of all clinical trials today do not meet their patient recruitment goals.

The present embodiments also force a new predictive analytical layer based on patient activity and urgency to help improve the patient engagement, recruitment and retention inefficiencies in the market, which is costing private insurance companies and Government entities like center for Medicare and Medicaid (CMMS (Centers for Medicare and Medicaid Services), etc.) to spend more on medication costs every year.

There has never been a behavioral platform and infrastructure before CAVII™ that includes patient behavioral criteria and adds patient engagement models based on behavioral characteristics, values, preferences that help create an optimized patient cohort based not only on clinical but also on behavioral criteria. This is the industry's first analytics-driven, predictive system that proactively profiles and segments patients based on patient values, attributes, behavioral drivers and is powered by a ASEMAP™ (Adaptive Self-Explicative Multiple Attribute Preference models) behavioral analytics algorithm. This algorithm is a powerful conjoint analysis and trade-off engine that specifically helps figure out what the patient truly wants and which benefit they prioritize over all the others

According to a first aspect of the present invention, a novel combination of structure and/or steps are provided for creating a patient object that is an intelligent combination of clinical, behavioral, social and financial models. Some of these come from digital health devices like FitBit, some from electronic medical data, some of them are answers to financial surveys and the behavioral index is based on powerful trade-off and conjoint analysis done on behavioral games, surveys and social media activity profiles shared by the patient.

According to a second aspect of the present invention, a novel combination of structure and/or steps is provided for predicting clinical urgency. For example the clinical urgency of an early stage diabetic patient is low as compared to the clinical urgency of an acutely diabetic patient trying to stave off the impending dialysis procedure due to his failing kidneys. This is completely different for a newly diagnosed cancer patient where the clinical urgency is very high as compared to a 10-year cancer survivor who is feeling less clinical urgency. This assessment is preferably done through quick intelligent surveys on a smart phone or other device. These clinical urgency indices are plotted as clinical urgency curves and used by the platform to analyze dynamically patient behavior and compliance. The system will offer Pharma companies or their intermediaries like CROs (Contract Research Organization) a real-time behavioral surveillance tool which can automate the process of the researchers calling/texting the patients many of whom may live hundreds of miles away from the research site. Giving them a smart phone with the system inside it will secure better engagement throughout the trials.

According to a third aspect of the present invention, a novel combination of steps is provided for behavioral prediction of likelihood to participate in a specific trial based on sophisticated trade-off analysis done on adaptive self-explication of multi-attribute preferences. These are done based on an ASEMAP™ tool and securing on-line patient responses to questions that are disease specific. Based on these the patients may be classified into specific behavioral personas (see FIG. 6). This is specific to diabetes, but equivalent behavioral personas are available for many other disease classes.

According to a fourth aspect of the present invention, a novel combination of structure and/or steps is provided for predicting engagement based on activity on social media and with wearable sensors like FitBit. For example, a person who meets his step goal of say 10,000 steps a day 90% of the time is predicted to be exhibiting a behavior persona that matches one of the five unique personas preferably modeled in the behavioral model system (see below). He/she will also score a 10/10 from a scoring perspective. His/her compliance with this goal over time creates a predictive score of how likely he is to meet his 10,000-step goal today.

Financial predictive index is based on a quick survey that gets answers to details around how the patient has paid for their health coverage, whether through their employer, Medicare, Medicaid, self-employed, supplemental insurance, or are they uninsured. The engagement index predicts based on their specific financial circumstances if they are more likely to sign up for trials. Regression models may be used that correlate type of insurance coverage with likelihood of participation in a trial.

In one preferred embodiment, a program embodied in a non-transitory computer readable medium provides a clinical trial patient recruitment system, said program comprising instructions causing at least one processor to perform:
receiving, via user input, identification and permission information from a plurality of users;
retrieving, from one or more clinical trial data repositories, clinical trial information;
retrieving, from one or more medical data repositories, clinical information corresponding to the plurality of users based on the identification and permission information as user clinical data;
retrieving, from one or more transactional data repositories, one or more of geographical, medical condition, medication, customer type, and time information corresponding to the plurality of users based on the identification and permission information as user behavioral data;
retrieving, from one or more of a mobile device and profile data repositories, one or more of dietary, fitness, activity level, and physiological monitoring information corresponding to the plurality of users based on the identification and permission information as user social data;
retrieving, from one or more financial data repositories, financial information corresponding to the plurality of users based on the identification and permission information as user financial data;
generating each of an urgency index, a behavioral index, an activity index, an affordability index, and a residual index corresponding to one or more clinical trials for one or more of the plurality of users based on the retrieved clinical trial information, user clinical data, user behavioral data, user social data, and user financial data;
generating a patient engagement prediction index for the one or more users based on a predetermined weighted relationship between the urgency index, the behavioral index, the activity index, the affordability index, and the residual index;
categorizing the one or more users into a plurality of groups corresponding to the one or more clinical trials based on the generated patient engagement prediction index;
forwarding solicitation data to users of one or more of the plurality groups for participating in the one or more clinical trials;
receiving participation data from one or more of the users of the one or more groups; and forwarding the participation data to one or more apparatuses corresponding to the one or more clinical trials.

In another preferred embodiment, a system for recruiting clinical trial patients, comprises:
memory; and
one or more processors configured to execute one or more programs stored on said memory, said programs including:
instructions for receiving, via user input, identification and permission information from a plurality of users;
instructions for retrieving, from one or more clinical trial data repositories, clinical trial information;
instructions for retrieving, from one or more medical data repositories, clinical information corresponding to the plurality of users based on the identification and permission information as user clinical data;
instructions for retrieving, from one or more transactional data repositories, one or more of geographical, medical condition, medication, customer type, and time information corresponding to the plurality of users based on the identification and permission information as user behavioral data;
instructions for retrieving, from one or more of a mobile device and profile data repositories, one or more of dietary, fitness, activity level, and physiological monitoring information corresponding to the plurality of users based on the identification and permission information as user social data;
instructions for retrieving, from one or more financial data repositories, financial information corresponding to the plurality of users based on the identification and permission information as user financial data;
instructions for generating each of an urgency index, a behavioral index, an activity index, an affordability index, and a residual index corresponding to one or more clinical trials for one or more of the plurality of users based on the retrieved clinical trial information, user clinical data, user behavioral data, user social data, and user financial data;
instructions for generating a patient engagement prediction index for the one or more users based on a predetermined weighted relationship between the urgency index, the behavioral index, the activity index, the affordability index, and the residual index;
instructions for categorizing the one or more users into a plurality of groups corresponding to the one or more clinical trials based on the generated patient engagement prediction index;
instructions for forwarding solicitation data to users of one or more of the plurality groups for participating in the one or more clinical trials;
instructions for receiving participation data from one or more of the users of the one or more groups; and
instructions for forwarding the participation data to one or more apparatuses corresponding to the one or more clinical trials.

Thus, the present invention creates and provides a single stream integrating multiple data sources that uniquely includes a patient's drivers of engagement behavior allowing for a far superior trial outcome.

### BRIEF DESCRIPTION OF THE DRAWINGS

Exemplary embodiments of the presently preferred features of the present invention will now be described with reference to the accompanying drawings.

FIG. 1 is a block diagram illustrating the clinical trial recruitment system in accordance with an embodiment of the invention.

FIG. 2 is a block diagram illustrating a software structure for implementing the clinical trial recruitment system in accordance with an embodiment of the invention.

FIG. 3 is an illustration of a patient capsule representing categories of information corresponding to a patient, according to the invention.

FIG. 4 is a block diagram illustrating an overall clinical trial engagement index.

FIG. 5 is a schematic view of a clinical urgency index for a particular disease according to an exemplary embodiment of the invention.

FIG. 6 is a diagram illustrating a grouping of patients based on index profiling according to an embodiment of the invention.

FIGS. 7A and 7B are process diagrams illustrating a decision identifying a consumer as a patient and non-patient and the retrieval of patient information according to an embodiment of the invention.

FIG. 8 is a block diagram illustrating a presently preferred hardware configuration system in accordance with an embodiment of the invention.

FIG. 9 is a block diagram illustrating a patient-specific software diagram for implementing the clinical trial recruitment system in accordance with an embodiment of the invention.

FIG. 10 is an illustration of a software diagram from the vantage point of a Contract Research Organization (CRO).

FIG. 11 illustrates a software diagram for a healthcare provider in a clinical research site.

FIG. 12 is an illustration of a software diagram for other healthcare intermediaries like the FDA and others who may seek a view of the trial progress.

FIG. 13 is an illustration of one way to combine behavioral and clinical biomarkers to create a more complete view of the patient.

### DETAILED DESCRIPTION OF THE PRESENTLY PREFERRED EXEMPLARY EMBODIMENTS

The present invention is directed to a system and method for predicting patient engagement and targeting better allocation of expensive recruitment resources. However, the present invention may find applicability in other devices/systems, such as engaging in energy saving programs as a customer/consumer of energy, a predisposition to acquire certain goods or services that have a retail value etc.

Briefly, the preferred embodiments of the present invention provide for Greater market access, proactive patient stratification into disease-specific behaviors that create cohorts ideally suited to be matched up to clinical studies and trials that may be of interest to pharma companies and medical devices. This is significant as only one of ten trials on average finish on time and only 1 in two patients recruited today stay in the trial throughout its duration.

Advantageous features according to the present invention include:
A system in which optimal candidates likely to engage on the trial(s) is predicted based on their behavioral attributes
Apparatus in which the right patients are targeted reducing process resource wastage, false starts and unnecessary attrition.
A process in which participation is optimized and patient satisfaction is increased.

A “device” in this specification may include, but is not limited to, one or more of, or any combination of processing device(s) such as, a cell phone, a Personal Digital Assistant, a smart watch or other body-borne device (e.g., glasses, pendants, rings, etc.), a personal computer, a laptop, a pad, a cloud-access device, and/or any device capable of sending/receiving messages to/from a local area network or a wide area network (e.g., the Internet), such as devices embedded in cars, trucks, aircraft, household appliances (refrigerators, stoves, thermostats, lights, electrical control circuits, the Internet of Things, etc.).

An “engine” is preferably a program that performs a core function for other programs. An engine can be a central or focal program in an operating system , subsystem, or application program that coordinates the overall operation of other programs. It is also used to describe a special-purpose program containing an algorithm that can sometimes be changed. The best known usage is the term search engine which uses an algorithm to search an index of topics given a search argument. An engine is preferably designed so that its approach to searching an index, for example, can be changed to reflect new rules for finding and prioritizing matches in the index. In artificial intelligence, for another example, the program that uses rules of logic to derive output from a knowledge base is called an inference engine.

As used herein, a “server” may comprise one or more processors, one or more Random Access Memories (RAM), one or more Read Only Memories (ROM), one or more user interfaces, such as display(s), keyboard(s), mouse/mice, etc. A server is preferably apparatus that provides functionality for other computer programs or devices, called “clients.” This architecture is called the client-server model, and a single overall computation is typically distributed across multiple processes or devices. Servers can provide various functionalities, often called “services”, such as sharing data or resources among multiple clients, or performing computation for a client. A single server can serve multiple clients, and a single client can use multiple servers. A client process may run on the same device or may connect over a network to a server on a different device. Typical servers are database servers, file servers, mail servers, print servers, web servers, game servers, application servers, and chat servers. The servers discussed in this specification may include one or more of the above, sharing functionality as appropriate. Client-server systems are most frequently implemented by (and often identified with) the request-response model: a client sends a request to the server, which performs some action and sends a response back to the client, typically with a result or acknowledgement. Designating a computer as “server-class hardware” implies that it is specialized for running servers on it. This often implies that it is more powerful and reliable than standard personal computers, but alternatively, large computing clusters may be composed of many relatively simple, replaceable server components.

The servers and devices in this specification typically use the one or more processors to run one or more stored “computer programs” and/or non-transitory “computer-readable media” to cause the device and/or server(s) to perform the functions recited herein. The media may include Compact Discs, DVDs, ROM, RAM, solid-state memory, or any other storage device capable of storing the one or more computer programs.

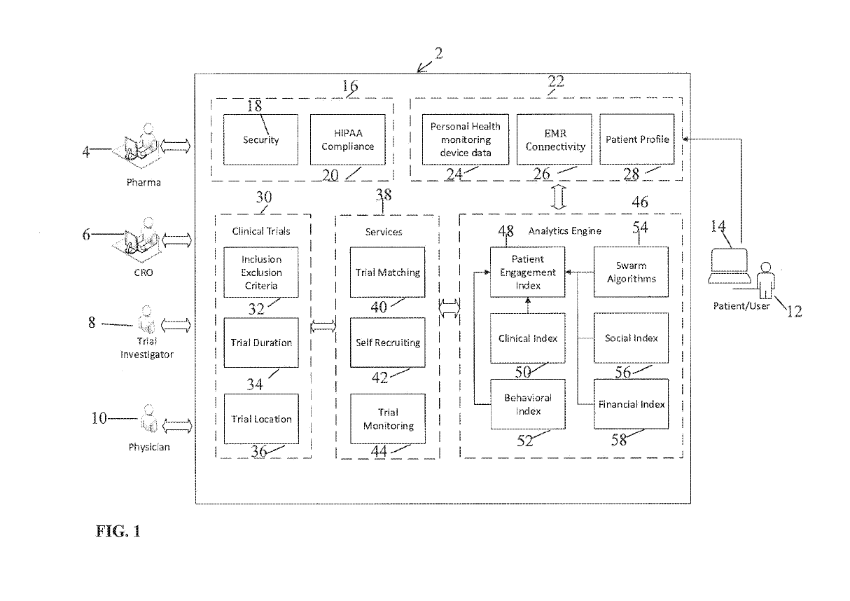

In FIG. 1, one or more servers 2 interfaces with one or more pharma clients 4, one or more CRO clients 6, one or more trial investigators 8, one or more physicians (or other health care providers such as nurses, physician's assistants, hospital staff, etc. 10, and one or more patents/users 12 through one or more patent devices/PCs/pads,/PDAs, etc. 14. The connection(s) may be via a Wide Area Network such as the Internet, and/or by way of a Local Area network such as an Ethernet network, or a combination of these. The connection may be wireless 9 e.g., WiFi) and/or wired.

In the one or more servers 2 are a number of modules/engines which preferably provide for: security/compliance 16 (preferably comprising one or more security engines 18 and one or more HIPPA compliance engines 20); patient information 22 (preferably comprising one or more personal health monitoring device data engines 24, one or more human API engines 26 (e.g., Electronic Medical Records (EMR) connectivity), and one or more patient profile engines 28); clinical trials information 30 (preferably comprising one or more inclusion/exclusion criteria engines 32, one or more trial duration engines 34, and one or more trial location engines 36; services 38 (preferably comprising one or more trial matching engines 40, one or more self-recruiting engines 42, and one or more trial monitoring engines 44); and at least one analytics engine 46 (preferably comprising one or more patient-engagement index engines 48, one or more clinical index engines 50, one or more behavioral index engines 52, one or more swarm algorithm engines 54, one or more social index engines 56, and one or more financial index engines 58).

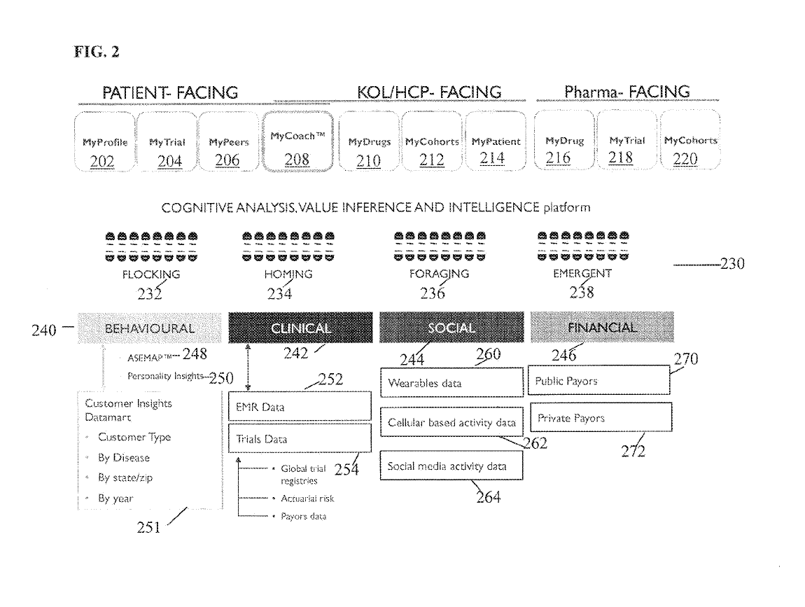

FIG. 2 is a functional schematic showing the various functions as they relate to the patient, the KOL/HCP (Key Opinion Leaders/Health Care Professionals) and the pharma entity (or entities). The patient, through one or more devices, has access to MyProfile module(s) 202, MyTrial module(s) 204, MyPeers module(s) 206, MyCoach module(s) 208, MyDrugs module(s) 210, MyCohorts module(s) 212 , MyPatient module(s) 214, MyDrug module(s) 216 (which may be the same or different from module(s) 210), MyTrial module(s) 218, and MyCohorts module(s) 220 (which may be the same or different from module(s) 210. Preferably, modules 202, 204, 206, and 208 are patient-facing modules; modules 208, 210, 212, and 214 are KOL/HCP facing; and modules 216, 218, and 220 are pharma-facing. A cognitive analysis, value inference, and intelligence platform (preferably comprising one or more engines) 230 has one or more modules/engines coordinating patient behaviors such as flocking engine 232, homing engine 234, foraging engine 236, and emergent engine 238.

Patient information may be stored in and/or analyzed by one or more memories and/or one or more engines comprising behavioral information 240, clinical information 242, social information 244, and financial information 246. The behavioral information 240 uses ASEMAP information 248 and personality insight information 250 to provide customer insight information to one or more memories/engines comprising a DataMart 251 having data preferably arranged by, at least, customer type, by disease, bat state/postal code, and by year. Clinical information 242 may be provided to one or more memories and/or one or more engines to provide EMR data 252 and trials data 254 (preferably comprising global trial registries information, actuarial risk information, and payer data information. Social information 244 may be provided to one or more memories and/or one or more engines to provide wearables data information 260, cellular based activity data information 262, and social media activity data information 264. Financial information 246 may be provided to one or more memories and/or one or more engines to provide public payer information 270 and private payer information 272.

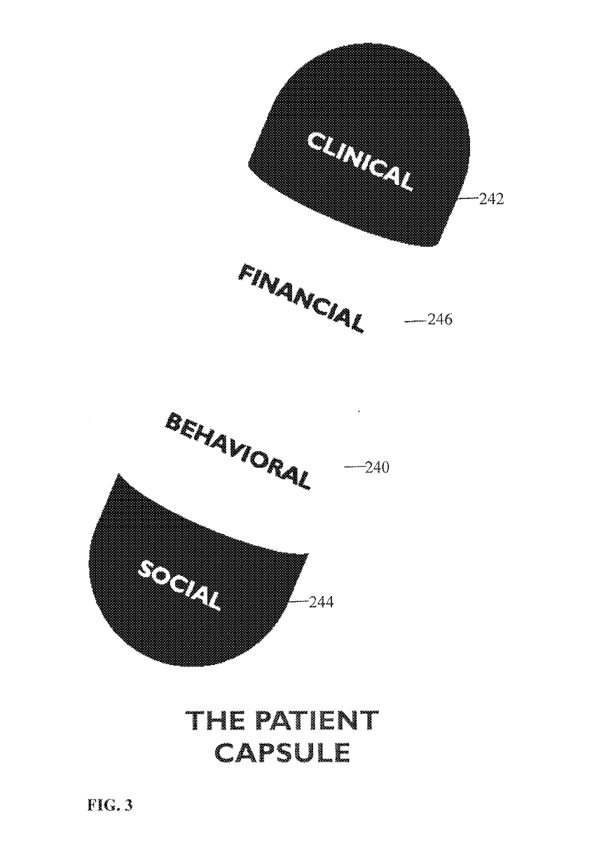

FIG. 3 is a notional view of the “patient capsule” information described above in FIG. 2, including the behavioral information 240, clinical information 242, social information 244, and financial information 246.

The ASEMAP™ tool may be used in a variety of scenarios, for example, in a Product/Service Attribute Improvement Trade-Off Exercise; a Product/Service Attribute/Feature Trade-Off Adaptive Conjoint Trade-off Exercise; a Product/Service Attribute Improvement Trade-Off Exercise; and in a Product/Service Attribute/Feature Trade-Off Exercise. In each scenario, at least two of the following functions are preferably performed: Every respondent reviews all the stimuli in providing their preferences for drivers of treatment, providing more robust, detailed, and actionable insights; Each respondent performs trade-offs between adaptively selected pairs of attributes, answering the question “Which one is more important to you? By how much more?”; Respondent then rank desirability of each of the levels on the attributes that are most important to that respondent; and Attribute importance is validated through reactions to product scenarios. Of course, many different scenarios involving many different exercises may be adapted depending on the trial, the respondents, and the drug, etc.

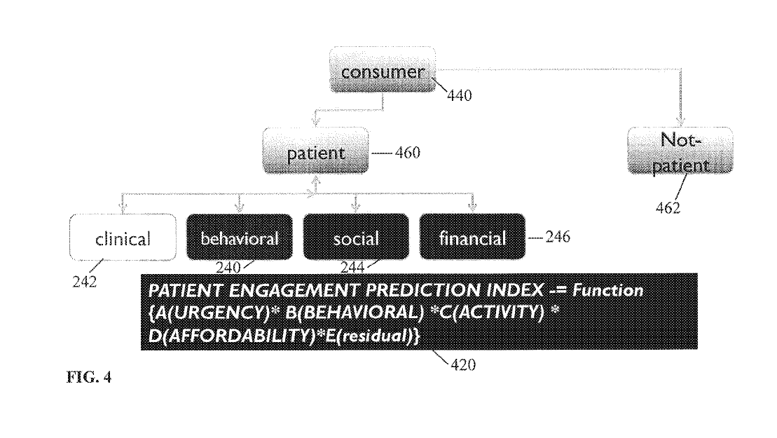

An embodiment using an ordinary least square regression model for calculating Patient Engagement Prediction Index PEPI) will now be described. Of course. many different (or a combination of) mathematical models may be used to provide appropriate patient engagement models, including ordinary least square regression, Bayesian models, agent based simulation, etc. The detail given here is the first starting point for the patent In FIG. 4, the patient engagement prediction index PEPI 420 is preferably calculate by a formula:

PEPI=A₀+A(clinical urgency)+B(behavioral urgency)+C(Social Media Activity Index)+D(Financial urgency index)+E(residual) (1)
Illustrative PEPI=0.05+0.6(Clinical)+0.35(Behavioral)+0.15(Social)+0.05(Financial)+E
Where A₀ is the intercept that shows a level of patient engagement when all independent variables are zero value.
Where A is the coefficient between PEPI and Clinical Urgency. A has values between 0 and 1 and the specific answer is 0.6 as solution for Clinical urgency showing how much of patient engagement can be explained by clinical behavior (example for diabetes this may be higher HbAlC values as a proxy for clinical urgency).
Where B is the coefficient between PEPI and Behavioral Urgency. B has values between 0 and 1 and the specific answer is 0.35 showing how much of patient engagement can be explained by patient behavioral modeling using ASEMAP.
Where C is the coefficient between PEPI and Social Media Urgency. C has values between 0 and 1 and the specific answer is 0.15 solution shows how much of patient engagement can be explained by social media.
Where D is the coefficient between PEPI and Financial Urgency. D has values between 0 and 1 and a 0.05 solution shows how much of patient engagement can be explained by financial conditions like having insurance, etc.
E in this case is the residual random leftover that cannot explain patient engagement at all
   Where a consumer 440 meets the PEPI threshold of greater than or equal to about 75, he/she becomes a patient 460, whose clinical, behavioral, social, and financial information is then used in the one or more trials. Otherwise, the consumer 440 is denoted as a non-patient 462.

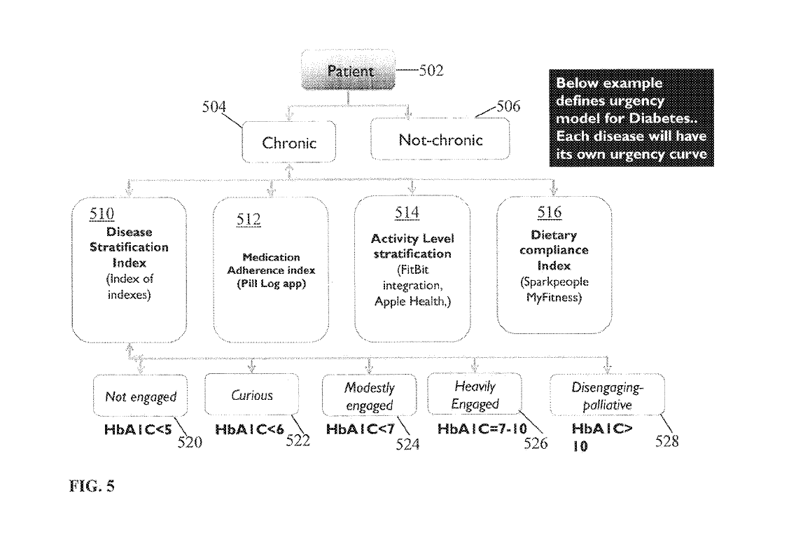

In FIG. 5, a preferred urgency model is defined for a patient 502. The patient's condition can be a chronic condition 504 or a non-chronic condition 506. If chronic, at least one disease stratification index (preferably an index of indexes) 510 is determined, along with at least one medication adherence index (e.g., a pill log application) 512, at least one activity level stratification index (e.g., FitBit integration and/or an Apple health application) 514, and a dietary compliance index (e.g., Sparkpeople application and/or MyFitness application) 516. The HbAlC (diabetes specific metric; below 5.5 are non-diabetic, above 5.5 are pre-diabetic, above 7 are diabetic) value is determined, and if below 5, the patient is not engaged 520, if below 6, the patient is curious 522, if below 7, the patient is modestly engaged 524, if from 7 to 10, the patient is heavily engaged 526, and of greater than 10, the patient is determined to be disengaging-palliative 528.

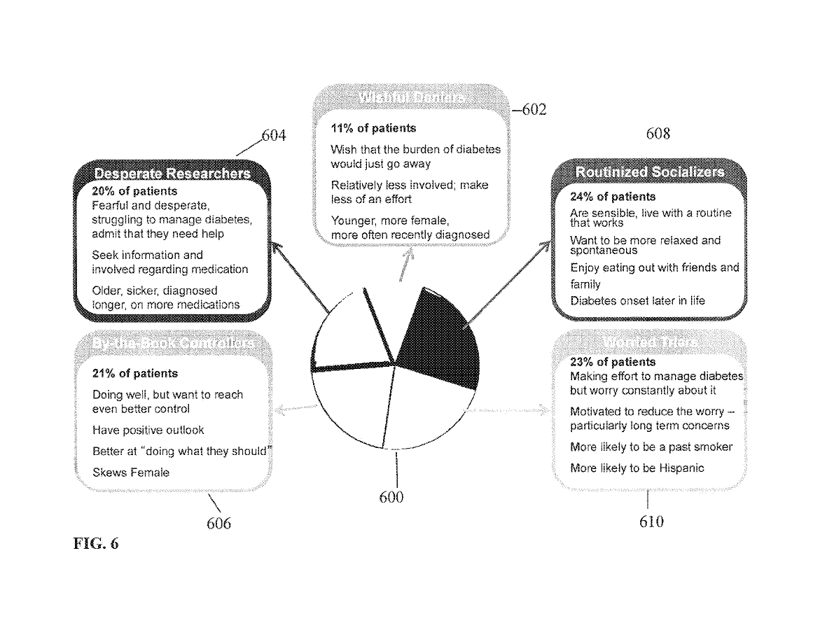

In FIG. 6, prospective patients 600 useful in clinical trials can be categorized as wishful deniers 602, comprising about 11 percent of patients who wish that the burden of a disease (e.g., diabetes) would just go away. Such patents are relatively less involved, and make less of an effort in the trial(s). They are often younger, more female, and are often recently diagnosed. Some patents are desperate researchers 604, comprising about 20 percent of patients. These patents are often fearful and desperate, struggling to manage the disease, and admit that they need help. They seek information and are relatively involved regarding their medication. They are often older, sicker, have been diagnosed for longer, and are on more medications. Then there are the by-the-book controller patients 606, which comprise about 21 percent of patients. They are typically doing well, but want to reach even better control over their disease. They typically have a positive outlook, and are better at doing what they should. The routinized socializer patients 608 comprise about 24 percent of patients, and are often sensible and live with a routine that works. They want to be more relaxed and spontaneous (e.g., going out with friends). The disease often sets in later in life. The worried-trier patients 610 comprise about 23 percent of patients. They are making efforts to manage their disease, but worry often about it. They are motivated to reduce the worry, particularly long term concerns. They may be a past smoker.

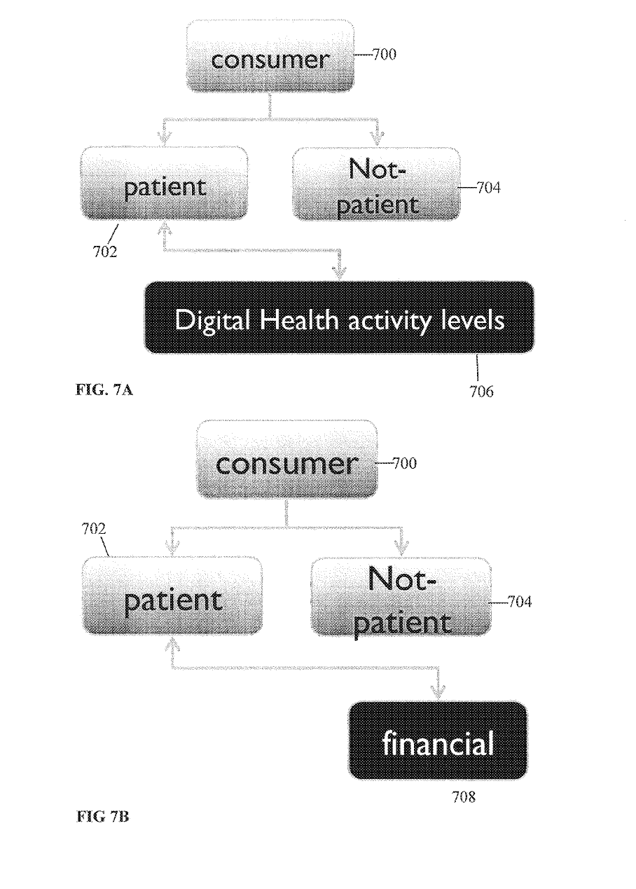

FIGS. 7A and 7B are process diagrams illustrating a decision identifying a consumer as a patient or a non-patient, and the retrieval of patient information. A consumer 700 will be determined to be a patient 702 (and not a non-patient 704), where the digital health activity levels (discussed above) 710 and/or the financial information (discussed above) 712 warrant that determination.

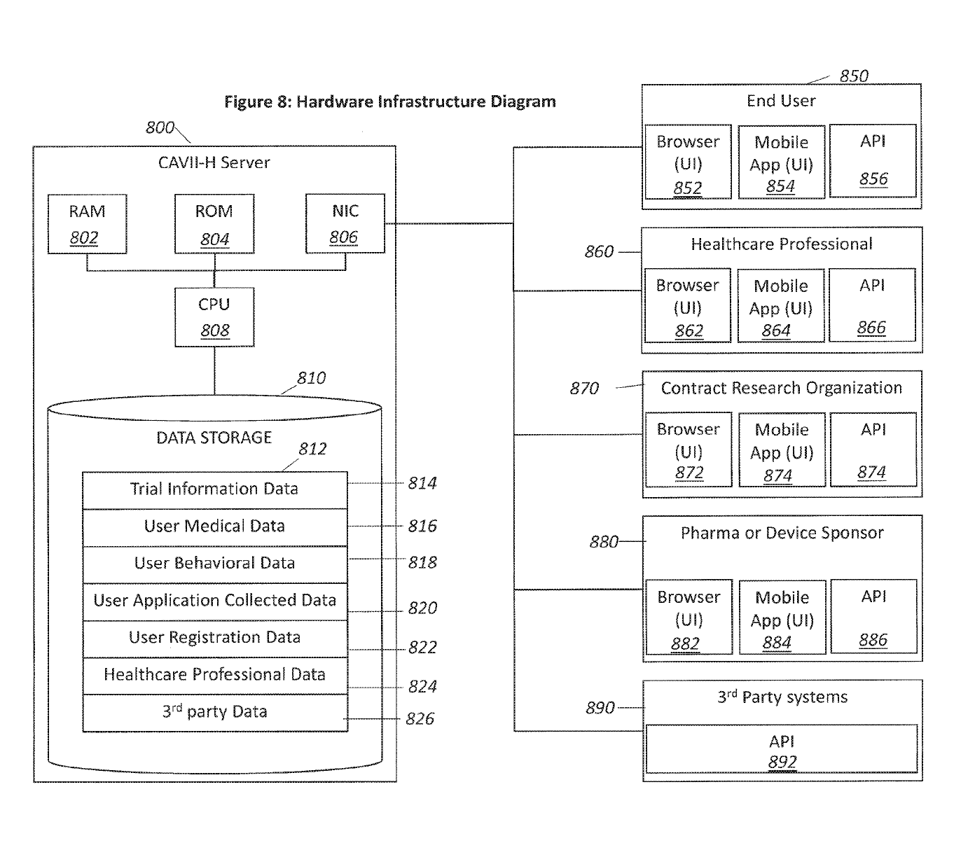

FIG. 8 is a hardware schematic diagram showing notable hardware features according to the preferred embodiments. It is useful to compare FIG. 8 with FIG. 1, which shows hardware and software features. In FIG. 8, the CAVII-H server 800 includes one or more RAM memories 802, one or more ROM memories 804 (typically storing computer program code for executing the functions described above and below), one or more network interface controllers (NIC) 806, and one or more central processing units (CPU), 808—each comprising one or more processors. In alternative embodiments, processing and storage functions may be shared among plural locations. One or more data base storage devices 810 store the data used in the embodiments described above and below. For example the one or more databases may store: trial information data 814; user medical data 816; user behavioral data 818; user application collected data 820; user registration data 822; healthcare professional data 824; and third party data 826.

The end user device or devices 850 preferably includes a user interface (e.g., a browser) 852, a mobile application API 854, and an API 856. Likewise, one or more healthcare professional devices 860 preferably includes a user interface (e.g., a browser) 862, a mobile application API 864, and an API 866. Further, one or more CRO devices 870 preferably includes a user interface (e.g., a browser) 872, a mobile application API 874, and an API 876. One or more pharma and/or device sponsors 880 preferably includes a user interface (e.g., a browser) 882, a mobile application API 884, and an API 886. One or more third party devices may include any and all of the above, for example, an API 892.

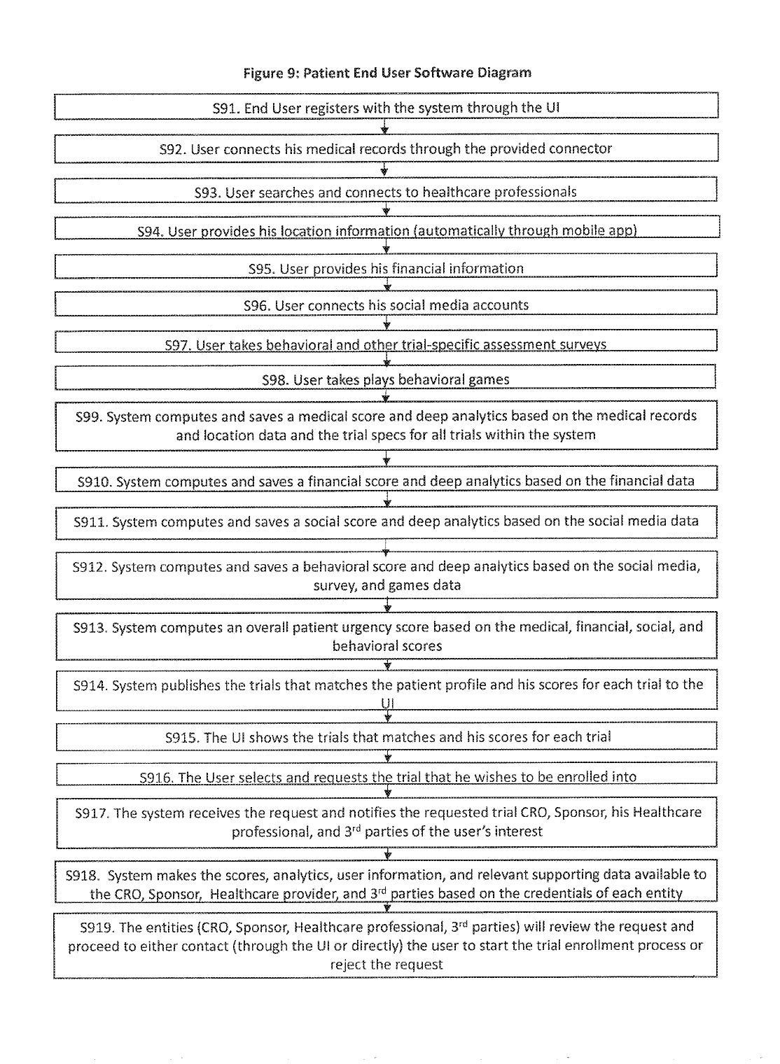

FIG. 9 is a block diagram illustrating a patient-specific software diagram for implementing the clinical trial recruitment system in accordance with an embodiment of the invention. Any or all (or any combination) of these steps may be taken, in order or any convenient order. In step S91, the end user registers with the system through the user interface (UI). In step S92, the user connects to/from his/her medical records through the provided connector. In step S93, the user searches and connects to healthcare professionals. In step S94, the user provides his/her location information (typically automatically through mobile app). In step S95, the user provides his/her financial information. In step S96, the user connects to/from his/her social media accounts. In step S97, the user takes behavioral and other trial-specific assessment surveys. In step S98, the user takes/plays behavioral games (if any). In step S99, the system server(s) computes and saves a medical score, and preferably deep analytics are performed based on the medical records and location data and the trial specs for all trials within the system server(s). In step S910, the system server(s) computes and saves a financial score and deep analytics are performed based on the financial data.

In step S911, the system server(s) computes and saves a social score and deep analytics are performed based on the social media data. In step S912, the system server(s) computes and saves a behavioral score and deep analytics are performed based on the social media, survey, and games data. In step S913, the system server(s) computes an overall patient urgency score based on the medical, financial, social, and behavioral scores. In step S914, the system server(s) publishes the trial(s) that matches the patient profile and his/her scores for each trial to the UI. In step S915, The UI shows the trials that match and his/her scores for each trial. In step S916, The user selects and requests the trial(s) that he/she wishes to be enrolled into. In step S917, the system server(s) receives the request and notifies the requested trial CRO, sponsor, his/her healthcare professional, and third parties of the user's interest. In step S918, the system server(s) makes the scores, analytics, user information, and relevant supporting data available to the CRO, sponsor, healthcare provider, and third parties based on the credentials of each entity. In step S919, the entities (CRO, sponsor, healthcare professional, third parties) will review the request and proceed to either contact the user (through the UI or directly) to start the trial enrollment process or reject the request.

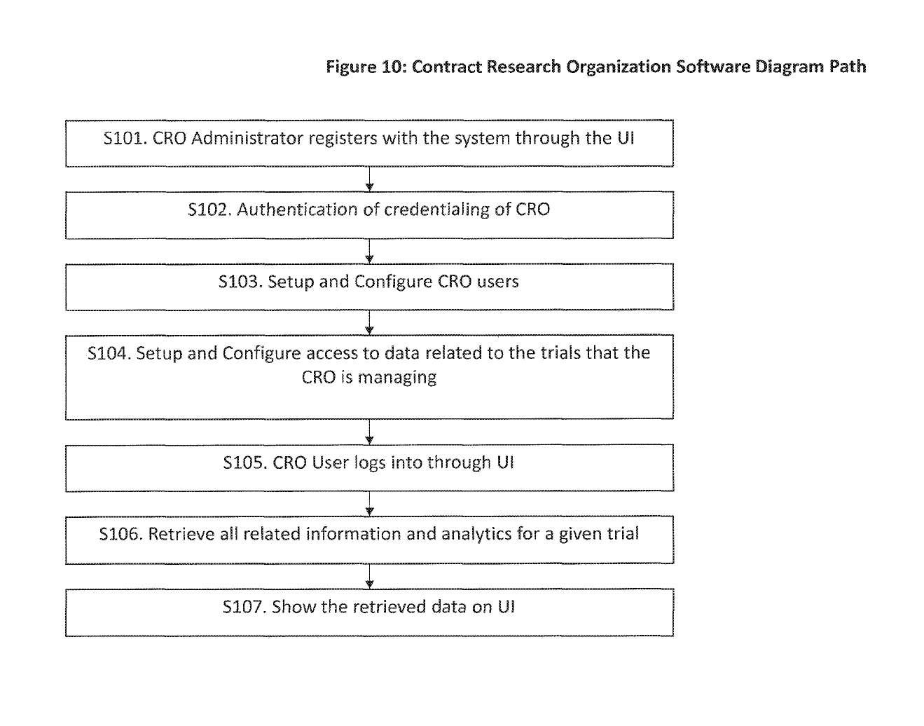

FIG. 10 is an illustration of a software diagram from the vantage point of a Contract Research Organization (CRO). Any or all (or any combination) of these steps may be taken, in order or any convenient order. In step S101, the sponsor administrator registers with the system server(s) through their UI. In step S102, authentication of and credentialing of the sponsor is performed by the system server(s). In step S103, setup and configuration of the sponsor's users is performed by the system server(s). In step S104, setup and configuration for access to the trial specifications data related to the trials that the sponsor is hosting is performed by the system server(s). In step S105, the sponsor user logs into the system server(s) through their UI. In step S106, the system server(s) retrieve all related information and analytics for a given trial. In step S107, the system server(s) shows the retrieved data on the UIs of the user, the sponsor, the CRO, the health care professional(s), and/or the third parties.

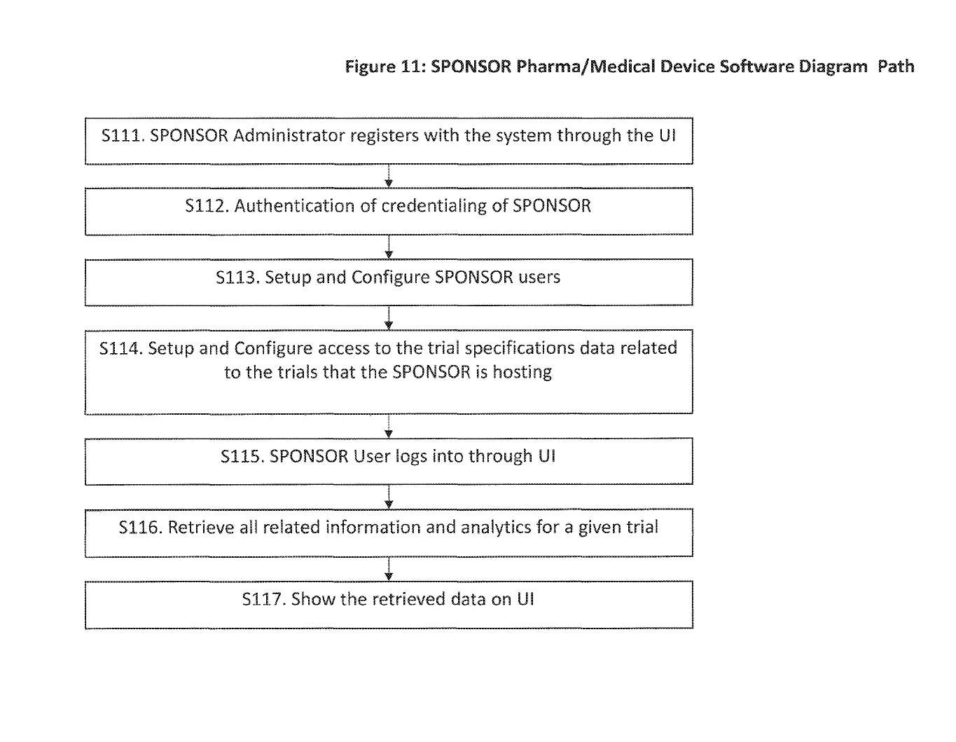

FIG. 11 illustrates a software diagram for a healthcare provider in a clinical research site. Any or all (or any combination) of these steps may be taken, in order or any convenient order. In step S111, the sponsor administrator registers with the system server(s) through the UI. In step S112, authentication and/or credentialing of the sponsor is performed by the system server(s). In step S113, setup and configuration of the sponsor's users is performed by the system server(s). In step S114, setup and/or configuration for access to the trial specifications data related to the trials that the sponsor is hosting is performed by the system server(s). In step S115, sponsor's user logs into the system server(s) through the users' UIs. In step S116, the system server(s) retrieve all related information and analytics for a given trial. In step S117, the system server(s) shows the retrieved data on the UIs of the user, the sponsor, the CRO, the health care professional(s), and/or the third parties.

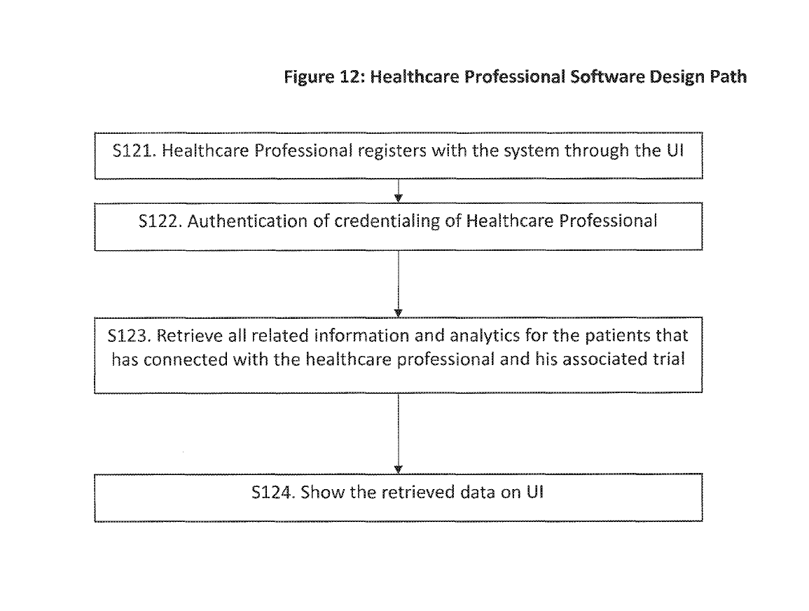

FIG. 12 is an illustration of a software diagram for other healthcare intermediaries like the FDA and others who may seek a view of the trial progress. Any or all (or any combination) of these steps may be taken, in order or any convenient order. In step S121, one or more healthcare professional(s) registers with the system server(s) through their UI. In step S122, authentication and/or of credentialing of the healthcare professional(s) is performed by the system server(s). In step S123, the system server(s) retrieve all related information and analytics for the patients that have connected with the healthcare professional and his/her associated trial. In step S124, the system server(s) shows the retrieved data on the UIs of the user, the sponsor, the CRO, the health care professional(s), and/or the third parties.

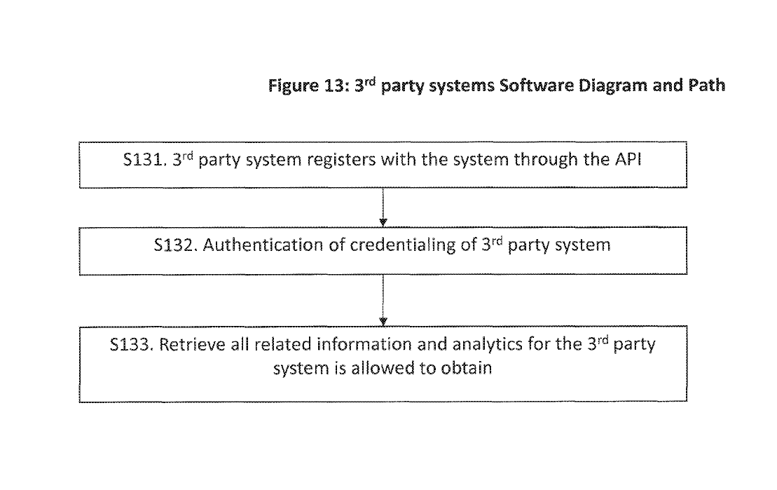

FIG. 13 is an illustration of one way to combine behavioral and clinical biomarkers to create a more complete view of the patient. Any or all (or any combination) of these steps may be taken, in order or any convenient order. In step S131, the third party server(s) registers with the system server(s) through their API(s). In step S132, authentication and/or of credentialing of the third party server(s) is performed by the system server(s). In step S133, the system server(s) retrieve all related information and analytics that the third party server(s) is allowed to obtain.

Thus, what has been described is a system which can eventually be a patient self-recruitment marketplace worldwide for clinical trials where buyers and sellers willingly transact with each other in a safe, secure way sharing highly sensitive information, accelerate drug development and secure regulatory approval.

While the present invention has been described with respect to what is presently considered to be the preferred embodiments, it is to be understood that the invention is not limited to the disclosed embodiments. To the contrary, the invention is intended to cover various modifications and equivalent arrangements included within the spirit and scope of the appended claims. The scope of the following claims is to be accorded the broadest interpretation so as to encompass all such modifications and equivalent structures and functions.

All U.S. and foreign patents and patent applications discussed above are hereby incorporated by reference into the Detailed Description of the Preferred Embodiments.

1-2. (canceled)

3. A method of self-recruitment of patients for a clinical trial by calculating a Patient Engagement Prediction Index (PEPI), comprising:
a plurality of patients suffering from a medical condition comprising potential clinical trial participants;
a plurality of clinical trials of potential value to the plurality of patients;
a computer system comprising a plurality of software engines and databases;
a self-recruitment computer application for use by patients;
wherein each patient engages the self-selection computer application, and the self-selection computer application receives, via patient input, each of:
identification and permission information from the plurality of patients;
clinical data on the medical condition of the patient;
behavioral and other trial-specific assessment information;
financial information, and
social media information;
wherein each of the each of the patient inputs is stored in one or more databases within the computer system;
wherein a software engine in the computer system computes a set of patient engagement scores for each patient based on the patient input data, where the scores comprise a clinical urgency index, a behavioral index, a social media index, and a financial index each patient;
wherein a software engine in the computer system receives, from each clinical trial, a set of clinical trial information, and wherein deep analytics determines trial-specific assessment criteria desired for each trial;
wherein a software engine is provided with a PEPI algorithm comprising coefficients for the clinical urgency score, a behavioral index score, a social media index score, and a financial index score, and wherein for each clinical trial, each PEPI coefficient is calculated according the inclusion and exclusion criteria for that trial;
wherein a software engine calculates a PEPI score for each patient for each clinical trial, wherein the PEPI is calculated from the patient engagement scores and the PEPI coefficients calculated for each clinical trial;
wherein the self-selection computer application presents a ranking of clinical trials of interest to the patient, wherein the ranking is determined by the PEPI score for that patient for each clinical trial;
and wherein the self-selection computer application permits a patient to select a specific clinical trial of interest.

4. The method of claim 3, wherein the patient participates in a selected clinical trial.

5. A method of self-recruitment of patients for a clinical trial by calculating a Patient Engagement Prediction Index (PEPI), comprising:
a plurality of patients suffering from a medical condition comprising potential clinical trial participants;
a plurality of clinical trials of potential value to the plurality of patients;
a computer system comprising a plurality of software engines and databases;
a self-recruitment computer application for use by patients;
wherein each patient engages the self-selection computer application, and the self-selection computer application receives, via patient input, each of:
identification and permission information from the plurality of patients and stores the identification and permission information in one or more databases within the computer system;
clinical data on the medical condition of the patient;
behavioral and other trial-specific assessment information;
financial information, and
social media information;
wherein a software engine in the computer system computes a set of patient engagement scores for each patient based on the patient input data, where the scores comprise a clinical urgency index, a behavioral index, a social media index, and a financial index each patient;
wherein a software engine in the computer system receives, from each clinical trial, a set of clinical trial information, and wherein deep analytics determines trial-specific assessment criteria desired for each trial;
wherein a software engine is provided with a PEPI algorithm according to formula (1):
PEPI=A₀+A(clinical urgency score)+B(behavioral urgency score)+C(Social Media Activity score)+D(Financial urgency score)+E(residual score) (1)
wherein for each clinical trial, each PEPI coefficient from A to E is calculated according trial-specific assessment criteria for that clinical trial;
wherein a software engine calculates a PEPI score for each patient for each clinical trial, wherein each PEPI score is calculated from the patient engagement scores and the PEPI coefficients calculated for that clinical trial;
wherein the self-selection computer application presents a ranking of clinical trials of interest to the patient, wherein the ranking is determined by the PEPI score for that patient for each clinical trial;
and wherein the self-selection computer application permits a patient to select a specific clinical trial of interest.

6. The method of claim 5, wherein the patient participates in a selected clinical trial.

7. The method of claim 3, wherein the self-selection computer application operates on a mobile computer, a laptop, or a workstation computer.

8. The method of claim 3, wherein at least one of the one or more databases storing patient inputs is within a server.

9. The method of claim 3, wherein the software engine in the computer system that computes the patient engagement scores operates on a server.
